(19) United States
(12) Patent Application Publication
(10) Pub. No.: US 2016/0136181 A1
(43) Pub. Date: May 19, 2016

(54) MICRORNA DOSING REGIMENS

(71) Applicant: Mirna Therapeutics, Inc., Austin, TX (US)

(72) Inventor: Sinil Kim, Austin, TX (US)

(73) Assignee: Mirna Therapeutics, Inc, Austin, TX (US)

(21) Appl. No.: 14/676,308

(22) Filed: Apr. 1, 2015

Related U.S. Application Data
(60) Provisional application No. 62/079,858, filed on Nov. 14, 2014.

(51) Int. Cl. A61K 31/573; A61K 31/7088; A61K 45/06; C12N 15/113

(52) U.S. Cl. CPC A61K 31/573; C12N 15/113; A61K 31/7088; A61K 45/06; C12N 2310/141

(57) ABSTRACT

A method of treating a subject, for example for a subject with a solid tumor or hematologic malignancy, can include administering a therapeutic treatment cycle to the subject, the cycle including daily microRNA mimic administrations on the first 3-7 consecutive days of the cycle followed by no microRNA administration on the next 7-21 consecutive days of the cycle.

### CROSS REFERENCE

This application claims the benefit of priority to U.S. Provisional Application No. 62/079,858, filed Nov. 14, 2014, and U.S. Provisional Application No. 61/973,332, filed Apr. 1, 2014, both of which are incorporated herein by reference in their entireties.

### SEQUENCE LISTING

The present application contains a Sequence Listing which has been submitted in ASCII format via EFS-Web and is hereby incorporated by reference in its entirety. Said ASCII copy, created, Apr. 1, 2015, is named “252_Sequence.txt” and is 2,264 bytes in size.

### FIELD OF THE INVENTION

The invention relates generally to therapeutic microRNA mimic dosing regimens. In some embodiments, the invention relates more particularly to therapeutic microRNA mimic dosing regimens for hematologic malignancies and/or solid tumors.

### BACKGROUND OF THE INVENTION

Micro-ribonucleic acids (microRNAs) belong to a class of small non-coding RNAs. They regulate many biological processes, including the cell cycle, cell growth and differentiation, stress response and apoptosis. Alterations in microRNA synthesis occur in human cancers and these are often linked to tumor development, progression and metastasis. Epigenetic alterations and mutations of microRNA expression may promote tumor formation as well as increased tumor aggressiveness, invasion, metastasis and resistance to chemotherapy and radiotherapy. It has been postulated that deregulation of microRNA synthesis, which regulates protein synthesis, is one of the most important factors implicated in cancer development.

These findings suggest novel ways of blocking cancer-related cell proliferation, by re-expression of microRNAs inhibited or silenced by cancer development or by inhibiting oncogenic microRNAs. This might be achieved by introducing molecules that mimic the expression of protective microRNAs that are down-regulated in cancer, or by introducing synthetic antisense molecules complementary to the microRNA of interest and which inhibit oncogenic microRNAs overexpressed in cancer cells (i.e. antagomiRs, anti-miRs).

One of the best-characterized microRNAs to date is microRNA-34 (miR-34). Human miR-34 comprises three family members: miR-34a, miR-34b and miR-34c. These miR-34 genes are frequently inactivated or expressed at reduced levels in numerous cancer types. miR-34a-c frequently functions downstream of p53 by regulating genes that induce cell cycle arrest, cellular senescence and apoptosis.

The re-introduction of miR-34a inhibits cancer cell growth both in vitro and in vivo. Therapeutic activity of miR-34a has been demonstrated in animal models of non-small cell lung cancer, prostate cancer, melanoma, pancreatic cancer and lymphoma, generally showing 50% to 83% tumor growth inhibition. In order to efficiently deliver miR-34a to tumors in vivo upon intravenous administration, Mirna Therapeutics has evaluated multiple existing delivery systems that are in pre-clinical development or have already entered clinical testing with other oligonucleotide therapeutics. Based on this systemic evaluation program, Mirna Therapeutics has selected a liposomal delivery formulation which is complexed with synthetically produced mimics of miR-34a, and which constitutes the therapeutic drug candidate, MRX34. Evaluations of efficacy in murine cancer models, microRNA bio-distribution and preliminary safety have been performed.

Nucleic acid delivery technologies are being developed in connection with various nucleic acids therapeutic candidates. One delivery technology is liposomes, for example amphoteric liposomes like Marina Biotech's SMARTICLES®. Amphoteric liposomes are a class of liposomes, which are pH dependent charge-transitioning particles that can provide for the delivery of a nucleic acid payload (e.g., siRNA, microRNA, antisense, etc.) to cells either by local or systemic administration. Amphoteric liposomes can be designed to release their nucleic acid payload within the target cell where the nucleic acid can then engage a number of biological pathways, and thereby exert a therapeutic effect.

ProNAi Therapeutics has used the NOV340 SMARTICLES® liposomal formulation encapsulating a single-stranded DNA that targets BCL2. With ProNAi's formulation 2 complete remission and 1 partial remission were observed out of 6 patients with either follicular lymphoma or diffuse large B-cell lymphoma. Out of 9 patients with evaluable safety information, the following drug-related adverse events were seen: nausea (8 pts); chills (6 pts); diarrhea (5 pts); fever, tumor pain, vomiting (5 pts each); and anorexia, back pain, fatigue (3 pts each). Most of these adverse events were of low grade and no grade 4 toxicity was observed.

ProNAi Therapeutics has completed a phase I study (ClinicalTrials.gov Identifier: NCT01191775) in Patients With Advanced Solid Tumors, and has an ongoing phase II study (ClinicalTrials.gov Identifier: NCT01733238) for Treatment of Relapsed or Refractory Non-Hodgkin's Lymphoma, both using a liposome encapsulated oligonucleotide (DNA Interference, or DNAi) drug substance that was administered by intravenous infusion once daily for 5 consecutive days of a 21-day cycle.

Tekmira Pharmaceuticals has used lipid nanoparticles which share some similarity with NOV340 SMARTICLES® to deliver oligonucleotides directed against PLK and found tumor responses in patients with adrenocortical carcinoma and neuroendocrine tumor.

As of March 2013, Mirna Therapeutics (Austin, Tex.) has completed the preclinical development program to support the manufacture of cGMP-materials and the conduction of IND-enabling studies for a miR-34-based supplementation therapy (MRX34). Mirna Therapeutics evaluated the toxicity as well as the pharmacokinetic profile of the formulation containing miR-34 mimic in non-GLP pilot studies using mice, rats and non-human primates. These experiments did not show adverse events at the predicted therapeutic levels of MRX34, as measured by clinical observations, body weights, clinical chemistries (including LFT, RFT and others), hematology, gross pathology, histopathology of select organs and complement (CH₅₀). In addition, miRNA mimics formulated in lipid nanoparticles do not induce the innate immune system as demonstrated in fully immunocompetent mice, rats, non-human primates, as well as human whole blood specimens. A more detailed review of the pre-clinical data is provided in Bader, Front Genet. 2012; 3:120. Clinical trials are ongoing and, as of Mar. 27, 2014, twenty-nine patients have been treated with MRX34, three at 10 mg/m², six at 20 mg/m², three at 33 mg/m², eight at 50 mg/m², seven at 70 mg/m², and two at 93 mg/m² on a twice weekly dosing schedule.

### SUMMARY OF THE INVENTION

The invention is based, at least in part, on the discovery that certain microRNA dosing regimens provide advantageous and unexpectedly superior therapies, for examples with (i) decreased toxicity, (ii) decreased side effects, and/or (iii) increased efficacy. In doing so, the invention provides improved methods for microRNA treatments of hematologic malignancies and/or solid tumors. In various embodiments, toxicity and efficacy results in humans can be surprisingly different from that obtained from animals, e.g., including mice and non-human primates. For example, daily×5 day dosing of MRX34 can be surprisingly less toxic and more effective than every other day dosing or twice weekly dosing of MRX34 in humans (i.e., despite the observation that every other day dosing or twice weekly dosing of MRX34 in animals had minimal toxicity and high efficacy).

The invention provides therapeutic microRNA dosing regimens for hematologic malignancies and/or solid tumors where the microRNAs are mimics of microRNAs involved in the hematologic malignancy and/or solid tumors being treated. For example, the microRNA can be a mimic of a miR-34 family member, or a mimic of another microRNA downregulated in a hematologic malignancy and/or solid tumors. The microRNA mimic is administered in one or more treatment cycles in which the microRNA is administered daily for a certain number of consecutive days, followed by a number of consecutive days without microRNA administration.

Accordingly, in various aspects, the invention provides a method of treating a subject comprising administering a therapeutic treatment cycle to the subject, the cycle including daily microRNA mimic administrations on the first 3-7 consecutive days of the cycle followed by no microRNA administration on the next 7-21 consecutive days of the cycle, thereby treating the subject.

In various aspects, the invention also provides a method for treating a subject comprising administering a therapeutically effective amount of a microRNA to the subject in treatment cycle including (i) 3-7 consecutive days of microRNA administration, followed by (ii) 7-21 days of without microRNA administration.

In addition to the number of consecutive day with and without microRNA administration, the particular microRNA dosing can be an important feature of the invention.

Accordingly, in various aspects, the invention also provides a method for treating a human subject having a cancer comprising administering a therapeutically effective amount of microRNA to the subject on 3-7 consecutive days of a 7-28 day treatment cycle, wherein the therapeutically effective amount comprises 20 mg/m² to 370 mg/m² (or 10 mg/kg).

In various aspects, the invention also provides a method for treating a human subject having a hematologic malignancy and/or solid tumor comprising administering a therapeutically effective amount of a miR-34a, miR-34b, or miR-34c mimic to the subject on 5 consecutive days of a 21 day treatment cycle, wherein the therapeutically effective amount comprises 20 mg/m² to 370 mg/m² (or 10 mg/kg).

In various embodiments, and of the aspects described herein can be combined with one or more of the features discussed below.

In various embodiments, the subject is a human. The subject can alternatively be a non-human primate, or other laboratory animal (e.g., mouse, rat, guinea pig, rabbit, pig, and the like). The subject can be a subject in need of a treatment in accordance with the present invention. For example, the subject can have a cancer, or more particularly a hematologic malignancy or solid tumor. Hematologic malignancies include, but are not limited to: leukemias (acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMoL), and other leukemias); lymphomas (Hodgkin's lymphomas (all four subtypes) and non-Hodgkin's lymphomas (all subtypes)); as well as myelomas. Certain embodiments can be specifically directed to one of these hematologic malignancies. The cancer can be a solid tumor. Solid tumors include, but are not limited to, hepatocellular carcinoma (HCC), non-small cell lung cancer (NSCLC), breast cancer, colorectal cancer, pancreatic cancer, and any cancer metastatic to the liver or bone marrow.

In some embodiments, the cancer is not a solid tumor (e.g., an advanced solid tumor). In some embodiments, the cancer is not a lymphoma, prostate cancer, or melanoma. In some embodiments, the cancer is not a lymphoma, for example a non-Hodgkin's lymphoma (e.g., relapsed or refractory non-Hodgkin's lymphoma).

In various embodiments, the microRNA mimic is formulated in a liposomal injectable suspension. Formulations are discussed further in the detail description below.

In various embodiments, the microRNA is a miR-34a, miR-34b, or miR-34c mimic. The microRNA can be a microRNA mimic of another microRNA downregulated in a hematologic malignancy, and for which a mimic of the microRNA is therapeutically effective.

In various embodiments, the microRNA is administered to the subject on the first 5 consecutive days followed by no microRNA administration on the next 16 consecutive days in a 21 day treatment cycle. In various embodiments, the examples can be modified to provide additional embodiments where (i) the therapeutically effective amount of the microRNA is administered to the subject on 3, 4, 5, 6, or 7 consecutive days of a 1, 2, 3, or 4 week treatment cycle, (ii) the therapeutically effective amount of the microRNA is administered to the subject on 5 consecutive days of a 2, 3, or 4 week treatment cycle, and (iii) the therapeutically effective amount of the microRNA is administered to the subject on 4, 5, or 6 consecutive days of a 3 week treatment cycle.

In various embodiments, the microRNA is a miR-34 family mimic comprising a sequence that is at least 80, 85, 90, or 95% identical to any one of SEQ ID NO:1-9. In various embodiments, the microRNA is a miR-34 family mimic comprising a sequence that is essentially identical to one of the seed or consensus sequences SEQ ID NO:4, 8 or 9 (e.g., having an identical sequence, or 1, 2, or 3 mismatches while retaining miR-34 function).

As discussed, the particular microRNA dosing can contribute to the unexpectedly superior results of the invention. In various embodiments, the microRNA is administered in an amount of 20 mg/m² to 370 mg/m² (or 10 mg/kg) per day. Example daily doses include: 20, 25, 30, 35, 40, 45, 50, 60, 70, 80, 90, 100, 125, 150, 175, 200, 225, 250, 275, 300, 325, 350, and 370 mg/m² per day. In some embodiments, the dose is not 120 mg/m² per day, or is less than 120 mg/m² per day (e.g., 100 mg/m² per day or less), or is greater than 120 mg/m² per day (e.g., 150 mg/m² per day or more).

The present invention can be used as a neo-adjuvant or adjuvant therapy (i.e., when used before or after complete removal by surgery or complete shrinkage by radiation therapy) or as a part of combination therapy (i.e., when used together with another cancer therapy). The present invention can also include additional therapeutics, for example when combined with an additional therapeutic to improve the efficacy of the microRNA mimic, or mitigate an undesired side effect of the microRNA mimic or the liposomal carrier.

In various embodiments, the method further comprises administering a therapeutically effective amount of a glucocorticoid, for example during the days of microRNA administration, for 1-5 days after the last microRNA administration, starting 1-3 days before the first microRNA administration, and/or starting 1-3 days before the first microRNA administration and during the days of microRNA administration. The method can further comprise administering the therapeutically effective amount of the glucocorticoid starting 1-3 days before the first microRNA administration, during the days of microRNA administration and for 1-5 days after the last microRNA administration. A therapeutically effective amount of the glucocorticoid can be 2-30 mg total daily dose of dexamethasone. A therapeutically effective amount of the glucocorticoid can be 10 mg total daily dose of dexamethasone. A therapeutically effective amount of the glucocorticoid can be administered 2-4 times daily. Examples of glucocorticoids include: cortisol (hydrocortisone), cortisone, prednisone, prednisolone, methylprednisolone, dexamethasone, betamethasone, triamcinolone, beclometasone, fludrocortisone acetate, deoxycorticosterone acetate, and aldosterone.

In various embodiments, the method further comprises administering a therapeutically effective amount of an immunosuppressive agent, with or without anticancer properties, for example during the days of microRNA administration, for 1-5 days after the last microRNA administration, starting 1-3 days before the first microRNA administration, and/or starting 1-3 days before the first microRNA administration and during the days of microRNA administration. The method can further comprise administering the therapeutically effective amount of an alkylating agent, an antimetabolite, mTOR (mammalian target of rapamycin) inhibitor, a polyclonal or monoclonal antibody, a cyclosporine, a mycophenolate, a TNF inhibitor, an activated complement inhibitor, or a calcineurin antagonist Immunosuppressive agents may be started 1-3 days before the first microRNA administration, during the days of microRNA administration and/or for 1-5 days after the last microRNA administration. A therapeutically effective amount of the immunosuppressive agents can be 0.1-1000 mg daily dose, depending on the specific agent.

Examples of alkylating agent include cyclophosphamide, nitrogen mustard (mechlorethamine) and busulfan. Examples of antimetabolite include methotrexate, azathioprine, mercaptopurine, and 5-fluorouracil. Examples of mTOR inhibitor include rapamycin (sirolimus), temsirolimus (CCI-779), deforolimus, everolimus, ridaforolimus. Examples of polyclonal antibody include antithymocyte immunoglobin (Atgam and Thymoglobuline), muromonab-CD3 (OKT3) and examples of monoclonal antibody include rituxan, obinutuzumab, basiliximab, daclizumab, and alemtuzumab. Examples of cyclosporine include cyclosporine A and cyclosporine G. Examples of mycophenolate include mycophenolate mofetil and mycophenolate sodium. Examples of TNF inhibitors include infliximab (Remicade®), adalimumab (Humira®), certolizumab pegol (Cimzia®), golimumab (Simponi®), and etenercept (Enbrel®). An example of complement inhibitors include eculizumab. Example of calcineurin inhibitor include cyclosporine, pimecrolimus and tacrolimus.

In various embodiments, the method further comprises administering the therapeutic treatment cycle to the subject two or more times. For example, treatment cycles can be will be repeated every 3 weeks (21 days) for hematologic malignancy patients based on toxicity and response. The schedule can continue as long as there is perceived benefit or until clinically significant disease progression. Similarly, in various embodiments, the method further comprises discontinuing therapy based upon one or more predetermined criteria (e.g., toxicity, undesired response, lack of a desired response, and the like).

In various aspects and embodiments, the invention includes a microRNA mimic for use according to any of the methods of the invention.

In various aspects and embodiments, the invention includes a pharmaceutical composition for use according to any of the methods of the invention.

In various aspects and embodiments, the invention includes administering a therapeutically effective amount of an immunosuppressive agent in combination with the microRNA.

Various aspects, embodiments, and features of the invention are presented and described in further detail below. However, the foregoing and following descriptions are illustrative and explanatory only and are not restrictive of the invention, as claimed.

### BRIEF DESCRIPTION OF THE DRAWINGS

FIG. 1 illustrates blood levels of MRX34 (ng/mL of blood) as a function of time from administration.

FIG. 2 illustrates a dosing timeline for a monkey study (results shown in FIGS. 3A-C).

FIGS. 3A-C illustrates blood levels of MRX34 by animal (3A) and by cohort (3B and 3C) as a function of time from administration.

FIGS. 4A-D illustrates correlation between nadir absolute neutrophil/platelet counts and MRX34 exposure.

FIG. 5 illustrates biodistribution of MRX34 in Non-Human Primates (N=3).

FIGS. 6A-B show the whole-blood pharmacokinetic profile of twice weekly MRX34 at 50 mg/m² dose level.

FIG. 7 shows the whole-blood pharmacokinetic profile of daily×5 MRX34.

FIGS. 8-10 present white blood cell gene expression for selected subjects 24 hours after first infusion with MRX34.

FIG. 11 presents the results for a subject showing confirmed partial response for HBV-HCC.

FIG. 12 presents the results for a subject showing prolonged stable disease in heavily pretreated SCLC.

FIG. 13A (baseline) and FIG. 13B (after cycle 2) present PET/CT scans after 2 cycles of 33 mg/m² QD×5 MRX34.

FIG. 14A (baseline) and FIG. 14B (after cycle 2) present PET/CT scans after 2 cycles of 33 mg/m² QD×5 MRX34.

### DETAILED DESCRIPTION OF THE INVENTION

The invention is based, at least in part, on the discovery that certain microRNA dosing regimens provide advantageous and unexpectedly superior therapies, for examples with (i) decreased toxicity, (ii) decreased side effects, and/or (iii) increased efficacy. In doing so, the invention provides improved therapeutics, for example for the treatment of hematologic malignancies and/or solid tumors. microRNA mimics, administration and dosing of microRNA, solid tumors and hematologic malignancies, as well as examples of the invention are discussed, in turn, below.

In various embodiments, toxicity and efficacy results in humans can be surprisingly different from that obtained from animals, e.g., including mice and non-human primates. For example, daily×5 day dosing of MRX34 can be surprisingly less toxic and more effective than every other day dosing or twice weekly dosing of MRX34 in humans (i.e., despite the observation that every other day dosing or twice weekly dosing of MRX34 in animals had minimal toxicity and high efficacy).

microRNA

microRNAs are small non-coding, naturally occurring RNA molecules that post-transcriptionally modulate gene expression and determine cell fate by regulating multiple gene products and cellular pathways (Bartel, Cell, 2004. 116(2):281-97). miRNAs interfere with gene expression by either degrading the mRNA transcript by blocking the protein translation machinery (Bartel, supra). miRNAs target mRNAs with sequences that are fully or merely partially complementary which endows these regulatory RNAs with the ability to target a broad but nevertheless specific set of mRNAs. To date, there are about 2,500 human annotated mature miRNA sequences with roles in processes as diverse as cell proliferation, differentiation, apoptosis, stem cell development, and immune function (Costinean et al., Proc Natl Acad Sci USA, 2006. 103(18):7024-9). Often, the misregulation of miRNAs can contribute to the development of human disease including cancer (Esquela-Kerscher et al., Nat Rev Cancer, 2006. 6(4):259-69; Calin et al., 2006. 6(11):857-66). miRNAs deregulated in cancer can function as bona fide tumor suppressors or oncogenes. A single miRNA can target multiple oncogenes and oncogenic signaling pathways (Forgacs et al., Pathol Oncol Res, 2001. 7(1):6-13), and translating this ability into a future therapeutic may hold the promise of creating a remedy that is effective against tumor heterogeneity. Thus, miRNAs have the potential of becoming powerful therapeutic agents for cancer (Volinia et al., Proc Natl Acad Sci USA, 2006. 103(7):2257-61; Tong et al., Cancer Gene Ther, 2008. 15(6):341-55) that act in accordance with our current understanding of cancer as a “pathway disease” that can only be successfully treated when intervening with multiple cancer pathways (Wiggins et al., Cancer Res, 2010. 70(14): 5923-5930.; Jones et al., Science, 2008. 321(5897):1801-6; Parsons et al., Science, 2008. 321(5897):1807-12).

In methods of the inventions, a specific synthetic microRNA (e.g., a microRNA mimic or similar synthetic oligonucleotide) is administered to a subject. In some embodiments, rather than an isolated cell, tissue, or culture thereof, the subject can be a mammal (e.g., a human or laboratory animal such as a mouse, rat, guinea pig, rabbit, pig, non-human primate, and the like). Administering a microRNA can include administering a microRNA vector, such as a viral vector, for example, to synthetically induce expression of a microRNA.

In various embodiments, the microRNA can be administered by methods such as injection or transfusion. microRNAs can be formulated in liposomes such as, for example, those described in U.S. Pat. Nos. 7,858,117 and 7,371,404; US Patent Application Publication Nos. 2009-0306194 and 2011-0009641. In some embodiments, the microRNA is formulated in an amphoteric liposomes, for example Marina Biotech's SMARTICLES®. Other delivery technologies are known in the art and available, including expression vectors, lipid or various ligand conjugates. Administering a microRNA can include administering a synthetic microRNA precursor, or synthetically inducing the expression of a microRNA precursor. Administering a microRNA can include administering a synthetic microRNA in hairpin form, for example a hairpin loop structure.

The microRNA can have a conventional naturally occurring sequences, as well as any chemically modified versions and sequence homologues thereof. microRNAs used in connection with the invention can be 7-130 nucleotides long, double stranded RNA molecules, either having two separate strands or a hairpin structure. For example, a microRNA can be 7, 8, 9, 10, 12, 13, 14, 15, 16, 17, 18, 19, 20, 21, 22, 23, 24, 25, 26, 27, 28, 29, 30, 7-30, 7-25, 15-30, 15-25, 17-30, or 17-25 nucleotides long. One of the two strands, which is referred to as the “guide strand”, contains a sequence which is identical or substantially identical to the seed sequence (nucleotide positions 2-9) of the parent microRNA sequence shown in the table below. “Substantially identical”, as used herein, means that at most 1 or 2 substitutions and/or deletions are allowed. In some embodiments, the guide strand comprises a sequence which is at least 80%, 85%, 90%, 95% identical to the respective full length sequence provided herein. The second of the two strands, which is referred to as a “passenger strand”, contains a sequence that is complementary or substantially complementary to the seed sequence of the corresponding given microRNA. “Substantially complementary”, as used herein, means that at most 1 or 2 mismatches and/or deletions are allowed. In some embodiments, the passenger strand comprises a sequence which is at least 60%, 65%, 70%, 75%, 80%, 85%, 90%, 95% identical to the complement of the respective full length sequence provided herein. In some embodiments, the oligonucleotide is a mimic of miR-34a, miR-34b, miR-34c, miR-449a, miR-449b, miR-449c, miR-192, or miR-215, or an analog or homolog thereof. In some embodiments, the oligonucleotide includes the seed sequence of one of these microRNAs.

TABLE 1
microRNA Sequences and Sequence
Identification Numbers

| microRNA | Sequence | SEQ ID NO: |
|---|---|---|
| miR-34a | UGGCAGUGUCUUAGCUGGUUGUU | SEQ ID NO: 1 |
| miR-34b | UAGGCAGUGUCAUUAGCUGAUUG | SEQ ID NO: 2 |
| miR-34c | AGGCAGUGUAGUUAGCUGAUUGC | SEQ ID NO: 3 |
| miR-34 | *GGCAGUGU*UUAGCUG*UUG* | SEQ ID NO: 4 |
| consensus | | |
| miR-449a | UGGCAGUGUAUUGUUAGCUGGU | SEQ ID NO: 5 |
| miR-449b | AGGCAGUGUAUUGUUAGCUGGC | SEQ ID NO: 6 |
| miR-449c | UAGGCAGUGUAUUGCUAGCGGCUGU | SEQ ID NO: 7 |
| miR-449 | UGGCAGUGUAUUG*UAGC*G*G | SEQ ID NO: 8 |
| consensus | | |
| miR-34/449 | GGCAGUG | SEQ ID NO: 9 |
| seed | | |

“*” denotes a deletion or any nucleotide(s). Seed sequences are shown in bold highlighting.

miR-34 is known to have anti-proliferative and potentially therapeutic activity. For example, when transfected with miR-34, cancer cell lines derived from patients with lung, liver, colon, pancreatic, breast, and prostate cancers as well as lymphoma and melanoma exhibit significantly reduced levels of proliferation and viability (Table 2, data not shown).

TABLE 2
Cancer Cell Lines in which MRX34 Demonstrates Anti-
proliferative Activity

| Lung | Liver | Colon | Pancreatic | Breast | Prostate | Mela- |
|---|---|---|---|---|---|---|
| Cancer | Cancer | Cancer | Cancer | Cancer | Cancer | noma |
| A549 | Hep3B | HCT116 | MIA- | BT-549 | PC3 | C32 |
| | | | PaCa2 | | | |
| H460 | HuH7 | SW48 | BxPC3 | MCF7 | DU145 | WM- |
| H1299 | HepG2 | HT29 | | T47D | PPC1 | 266-4 |
| H226 | C3A | LoVo | | MB-231 | LNCaP | |
| HCC-827 | SK-Hep1 | | | | | |
| | PLC/ | | | | | |
| | PRF/5 | | | | | |

The anti-proliferative activity of MRX34 results from its ability to inhibit cell cycle progression and induce apoptosis in cancer cells. miR-34 also inhibits sphere and colony formation of cancer stem cell-enriched populations Intravenous injections of liposome-formulated miR-34 inhibit the growth of mature tumors in mouse models of liver, lung, and prostate cancers as well as a model of lymphoma. Efficacy studies have been performed at multiple institutions by a variety of scientists, which demonstrates the robust therapeutic activity of miR-34 (Table 3, data not shown).

TABLE 3
Mouse Models of Cancer Used to Demonstrate Therapeutic Activity of
Systemically Delivered MRX34

| Cancer | | | |
|---|---|---|---|
| Type | Model | Result | Study Site |
| Liver | Hep3B orthotopic | Regression of mature | Mirna Therapeutics |
| Cancer | xenograft | tumors | |
| | HuH7 orthotopic | Regression of mature | Mirna Therapeutics |
| | xenograft | tumors | |
| Lung | H460 xenograft | Inhibition of tumor growth | BIOO Scientific |
| Cancer | A549 xenograft | Inhibition of tumor growth | BIOO Scientific |
| | Oncogenic kRAS GEMM | Inhibition of tumor growth | Yale University |
| | p53 null/oncogenic kRAS | Inhibition of tumor growth | Yale University |
| | GEMM | | |
| Prostate | LAPC9 orthotopic | Inhibition of tumor growth; | MD Anderson |
| Cancer | | Inhibition of metastasis | Cancer Center |
| | | | (MDACC) |
| | LAPC4 orthotopic | Inhibition of tumor growth | MDACC |
| | PC3 orthotopic | Inhibition of tumor growth | MDACC |
| | PPC-1 xenograft | Inhibition of tumor growth | BIOO Scientific |
| Lymphoma | U2932 xenograft | Inhibition of tumor growth | University of |
| | | | Zurich |

microRNAs can be chemically modified, for example, synthetic oligonucleotides may have a 5′ cap on the passenger strand (e.g., NH₂—(CH₂)₆—O—) and/or a mismatch at the first and/second nucleotide of the same strand. Other possible chemical modifications can include backbone modifications (e.g., phosphorothioate, morpholinos), ribose modifications (e.g., 2′-OMe, 2′-Me, 2′-F, 2′-4′-locked/bridged sugars (e.g., LNA, ENA, UNA) as well as nucleobase modifications (see, e.g., Peacock et al, 2011. J Am Chem Soc., 133(24):9200-9203. In certain embodiments, microRNAs have modifications as described in U.S. Pat. No. 7,960,359 and US Patent Application Publication Nos. 2012-0276627 and 2012-0288933.

In various embodiments, the microRNA is not a DNAi oligonucleotide.

In some embodiments, the microRNA is between 17 and 30 nucleotides in length and comprises (i) a microRNA region having a sequence from 5′ to 3′ that is at least 80% identical to at least one of SEQ ID NO:1-4, and (ii) a complementary region having a sequence from 5′ to 3′ that is 60-100% complementary to the microRNA region.

In some embodiments, the microRNA comprises a sequence that is at least 80, 85, 90, 95, or 100% identical to at least one of SEQ ID NO:1-4.

In some embodiments, the microRNA comprises a single polynucleotide or a double stranded polynucleotide. In some embodiments, the microRNA comprises a hairpin polynucleotide.

In some embodiments, the microRNA is between 17 and 30 nucleotides in length and comprises (i) a first polynucleotide having a sequence with at least 80% identical to at least one of SEQ ID NO:1-4; and (ii) a separate second polynucleotide having a sequence from 5′ to 3′ that is 60-100% complementary to the first polynucleotide.

In some embodiments, the microRNA is between 17 and 30 nucleotides in length and comprises one or more of the following (i) a replacement group for phosphate or hydroxyl of the nucleotide at the 5′ terminus of the complementary strand of the RNA molecule; (ii) one or more sugar modifications in the first or last 1 to 6 residues of the complementary region; or (iii) noncomplementarity between one or more nucleotides in the last 1 to 5 residues at the 3′ end of the complementary region and the corresponding nucleotides of the microRNA region.

In some embodiments, the microRNA is between 17 and 30 nucleotides in length and comprises (i) at least one modified nucleotide that blocks the 5′ OH or phosphate at the 5′ terminus, wherein the at least one nucleotide modification is an NH2, biotin, an amine group, a lower alkylamine group, an acetyl group or 2′oxygen-methyl (2′O-Me) modification; or (ii) at least one ribose modification selected from 2′F, 2′NH₂, 2′N₃, 4′thio, or 2′O—CH₃.

In some embodiments, the microRNA is between 17 and 30 nucleotides in length and comprises (i) a first polynucleotide having a sequence with at least 80% identical to at least one of SEQ ID NO:1-4; (ii) a separate second polynucleotide having a sequence from 5′ to 3′ that is 60-100% complementary to the first polynucleotide; and (iii) a lower alkylamine group at the 5′ end of the complementary strand.

In some embodiments, the microRNA is between 17 and 30 nucleotides in length and comprises (i) a first polynucleotide having 100% identical to at least one of SEQ ID NO:1-4; (ii) a separate second polynucleotide having a sequence from 5′ to 3′ that is 100% complementary to the first polynucleotide; and (iii) a lower alkylamine group at the 5′ end of the complementary strand.

### Hematologic Malignancies and Solid Tumors

The invention provides methods for treating cancer cells and/or tissue, including cancer cells and/or tissue in a subject, or in vitro treatment of isolated cancer cells and/or tissue. If in a subject, the subject to be treated can be an animal, e.g., a human or laboratory animal. Cancer can be caused by malignant tumors formed by an abnormal growth of cells and tissue leading to organ failure, and generally falls into two categories: solid and hematological cancers.

Solid tumors are neoplasms (new growth of cells) or lesions (damage of anatomic structures or disturbance of physiological functions) formed by an abnormal growth of body tissue cells other than blood, bone marrow or lymphatic cells. A solid tumor consists of an abnormal mass of cells which may stem from different tissue types such as liver, colon, breast, or lung, and which initially grows in the organ of its cellular origin. However, such cancers may spread to other organs through metastatic tumor growth in advanced stages of the disease.

In contrast, hematological tumors are cancer types affecting blood, bone marrow, and lymph nodes. Hematological tumors may derive from either of the two major blood cell lineages: myeloid and lymphoid cell lines. The myeloid cell line normally produces granulocytes, erythrocytes, thrombocytes, macrophages, and mast cells, whereas the lymphoid cell line produces B, T, NK and plasma cells. Lymphomas, lymphocytic leukemias, and myeloma are derived from the lymphoid line, while acute and chronic myelogenous leukemia, myelodysplastic syndromes and myeloproliferative diseases are myeloid in origin. As blood, bone marrow, and lymph nodes are intimately connected through the immune system, a disease affecting one haematological system may affect the two others as well.

Further to the discussion in the summary section above, in various embodiments of the invention hematologic malignancies include, but are not limited to, leukemias (Acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMoL), and other leukemias); lymphomas (Hodgkin's lymphomas (all four subtypes) and non-Hodgkin's lymphomas (all subtypes)), as well as myelomas.

The subject being treated may have been diagnosed with cancer. The subject may have locally advanced, unresectable, or metastatic cancer and/or may have failed a prior first-line therapy. In various embodiments, the cancer is liver cancer (e.g., hepatocellular carcinoma, HCC). In various embodiments, the liver cancer (e.g., HCC) can be intermediate, advanced, or terminal stage. The liver cancer (e.g., HCC) can be metastatic or non-metastatic. Liver cancer can include a liver tumor resulting from the metastasis of a non-liver cancer, to the liver. The liver cancer (e.g., HCC) can be resectable or unresectable. The liver cancer (e.g., HCC) can comprise a single tumor, multiple tumors, or a poorly defined tumor with an infiltrative growth pattern (into portal veins or hepatic veins). The liver cancer (e.g., HCC) can comprise a fibrolamellar, pseudoglandular (adenoid), pleomorphic (giant cell), or clear cell pattern. The liver cancer (e.g., HCC) can comprise a well differentiated form, and tumor cells resemble hepatocytes, form trabeculae, cords, and nests, and/or contain bile pigment in cytoplasm. The liver cancer (e.g., HCC) can comprise a poorly differentiated form, and malignant epithelial cells are discohesive, pleomorphic, anaplastic, and/or giant. In some embodiments, the liver cancer (e.g., HCC) is associated with hepatitis B, hepatitis C, cirrhosis, or type 2 diabetes.

In some embodiments, the cancer is not a solid tumor (e.g., an advanced solid tumor). In some embodiments, the cancer is not a lymphoma, prostate cancer, or melanoma. In some embodiments, the cancer is not a lymphoma, for example a non-Hodgkin's lymphoma (e.g., relapsed or refractory non-Hodgkin's lymphoma).

microRNA Dosing and Administration

The invention provides therapeutic microRNA dosing regimens for hematologic malignancies and/or solid tumors where the microRNAs are mimics of microRNAs involved in the hematologic malignancy and/or solid tumors being treated. In various embodiments, the microRNA is not a DNAi oligonucleotide. The microRNA mimic is administered in one or more treatment cycles in which the microRNA mimic is administered daily for a certain number of consecutive days, followed by a number of consecutive days without microRNA administration.

The invention provides methods of treating a subject comprising administering a therapeutic treatment cycle to the subject, the cycle including daily microRNA mimic administrations on the first 3-7 consecutive days of the cycle followed by no microRNA administration on the next 7-21 consecutive days of the cycle, thereby treating the subject.

The invention also provides methods for treating a subject comprising administering a therapeutically effective amount of a microRNA to the subject in treatment cycle including (i) 3-7 consecutive days of microRNA administration, followed by (ii) 7-21 days of without microRNA administration.

The invention also provides methods for treating a human subject having a cancer comprising administering a therapeutically effective amount of microRNA to the subject on 3-7 consecutive days of a 7-28 day treatment cycle, wherein the therapeutically effective amount comprises 20 mg/m² to 370 mg/m² (or 10 mg/kg).

The invention also provides methods for treating a human subject having a hematologic malignancy and/or solid tumor comprising administering a therapeutically effective amount of a miR-34a, miR-34b, or miR-34c mimic to the subject on 5 consecutive days of a 21 day treatment cycle, wherein the therapeutically effective amount comprises 20 mg/m² to 370 mg/m² (or 10 mg/kg).

Example daily doses include: 20, 25, 30, 35, 40, 45, 50, 60, 70, 80, 90, 100, 125, 150, 175, 200, 225, 250, 275, 300, 325, 350, and 370 mg/m² per day. In some embodiments, the dose is not 120 mg/m² per day, or is less than 120 mg/m² per day (e.g., 100 mg/m² per day or less), or is greater than 120 mg/m² per day (e.g., 150 mg/m² per day or more). Doses are given in mg of microRNA. In various embodiments, the microRNA mimic is formulated in a liposomal injectable suspension. microRNA mimics can be administered intravenously as a slow-bolus injection at 20 mg/m² to 370 mg/m² (or 10 mg/kg) per day.

In various embodiments the microRNA is administered to the subject in 3, 4, 5, 6, or 7 daily doses over a single week (7 days). In various embodiments the microRNA is administered for: 1 week, 1 week with 1 week off (total 14 days); 2 weeks (total 14 days); 3 consecutive weeks (total 21 days); 2 weeks with 1 week off (total 21 days); 1 week with 2 weeks off (total 21 days); 4 consecutive weeks (total 28 days); 3 consecutive weeks with 1 week off (total 28 days); 2 weeks with 2 weeks off (total 28 days); 1 week with 3 consecutive weeks off (total 28 days).

In various embodiments, the microRNA is administered to the subject on the first 5 consecutive days followed by no microRNA administration on the next 16 consecutive days in a 21 day treatment cycle. The use of such embodiments is discussed in further detail in the examples below, which a person of ordinary skill will recognize as a basis for other variations in accordance with the invention. For example, the embodiments in the examples can be modified to other embodiments where (i) the therapeutically effective amount of the microRNA is administered to the subject on 3, 4, 5, 6, or 7 consecutive days of a 1, 2, 3, or 4 week treatment cycle, (ii) the therapeutically effective amount of the microRNA is administered to the subject on 5 consecutive days of a 2, 3, or 4 week treatment cycle, and (iii) the therapeutically effective amount of the microRNA is administered to the subject on 4, 5, or 6 consecutive days of a 3 week treatment cycle.

The present invention can be used as an adjuvant therapy (i.e., when combined with another cancer therapy). The present invention can also include additional therapeutics, for example when combined with an additional therapeutic to improve the efficacy of the microRNA mimic, or mitigate an undesired side effect of the microRNA mimic

In various embodiments, the method further comprises administering a therapeutically effective amount of a glucocorticoid, for example during the days of microRNA administration, for 1-5 days after the last microRNA administration, starting 1-3 days before the first microRNA administration, and/or starting 1-3 days before the first microRNA administration and during the days of microRNA administration. The method can further comprise administering the therapeutically effective amount of the glucocorticoid starting 1-3 days before the first microRNA administration, during the days of microRNA administration and for 1-5 days after the last microRNA administration. A therapeutically effective amount of the glucocorticoid can be 2-30 mg total daily dose of dexamethasone. A therapeutically effective amount of the glucocorticoid can be 10 mg total daily dose of dexamethasone. A therapeutically effective amount of the glucocorticoid can be administered 2-4 times daily. Examples of glucocorticoids include: Cortisol (hydrocortisone), Cortisone, Prednisone, Prednisolone, Methylprednisolone, Dexamethasone, Betamethasone, Triamcinolone, Beclometasone, Fludrocortisone acetate, Deoxycorticosterone acetate, and Aldosterone.

In various embodiments, the method further comprises administering a therapeutically effective amount of an immunosuppressive agent, with or without anticancer properties, for example during the days of microRNA administration, for 1-5 days after the last microRNA administration, starting 1-3 days before the first microRNA administration, and/or starting 1-3 days before the first microRNA administration and during the days of microRNA administration. The method can further comprise administering the therapeutically effective amount of an alkylating agent, an antimetabolite, mTOR (mammalian target of rapamycin) inhibitor, a polyclonal or monoclonal antibody, a cyclosporine, a mycophenolate, a TNF inhibitor, an activated complement inhibitor, or a calcineurin antagonist Immunosuppressive agents may be started 1-3 days before the first microRNA administration, during the days of microRNA administration and/or for 1-5 days after the last microRNA administration. A therapeutically effective amount of the immunosuppressive agents can be 0.1-1000 mg daily dose, depending on the specific agent.

Examples of alkylating agent include cyclophosphamide, nitrogen mustard (mechlorethamine) and busulfan. Examples of antimetabolite include methotrexate, azathioprine, mercaptopurine, and 5-fluorouracil. Examples of mTOR inhibitor include rapamycin (sirolimus), temsirolimus (CCI-779), deforolimus, everolimus, ridaforolimus. Examples of polyclonal antibody include antithymocyte immunoglobin (Atgam and Thymoglobuline), muromonab-CD3 (OKT3) and examples of monoclonal antibody include rituxan, obinutuzumab, basiliximab, daclizumab, and alemtuzumab. Examples of cyclosporine include cyclosporine A and cyclosporine G. Examples of mycophenolate include mycophenolate mofetil and mycophenolate sodium. Examples of TNF inhibitors include infliximab (Remicade®), adalimumab (Humira®), certolizumab pegol (Cimzia®), golimumab (Simponi®), and etenercept (Enbrel®). An example of complement inhibitors include eculizumab. Example of calcineurin inhibitor include cyclosporine, pimecrolimus and tacrolimus.

In various embodiments, the method further comprises administering the therapeutic treatment cycle to the subject two or more times. For example, treatment cycles can be will be repeated every 3 weeks (21 days) for hematologic malignancy patients based on toxicity and/or response. The schedule can continue as long as there is perceived benefit or until clinically significant disease progression. Similarly, in various embodiments, the method further comprises discontinuing therapy based upon one or more predetermined criteria (e.g., toxicity, undesired response, lack of a desired response, and the like). The efficacy of treatment can be assessed by the clinically accepted Response Criteria for the particular indication. Such Response Criteria are well known in the art, and can be applied in the various embodiments of the invention.

In various embodiments, the treatment reduces the size and/or number of the cancer tumor(s); prevent the cancer tumor(s) from increasing in size and/or number; and/or prevent the cancer tumor(s) from metastasizing.

microRNA can be delivered locally or systemically. In the methods of the invention, administration is not necessarily limited to any particular delivery system and may include, without limitation, parenteral (including subcutaneous, intravenous, intramedullary, intraarticular, intramuscular, or intraperitoneal injection), rectal, topical, transdermal, or oral (for example, in capsules, suspensions, or tablets). Administration to an individual may occur in a single dose or in repeat administrations, and in any of a variety of physiologically acceptable salt forms, and/or with an acceptable pharmaceutical carrier and/or additive as part of a pharmaceutical composition. Physiologically acceptable salt forms and standard pharmaceutical formulation techniques, dosages, and excipients are well known to persons skilled in the art (see, e.g., Physicians' Desk Reference (PDR®) 2005, 59^{th} ed., Medical Economics Company, 2004; and Remington: The Science and Practice of Pharmacy, eds. Gennado et al. 21th ed., Lippincott, Williams & Wilkins, 2005). Further description and embodiments of combination therapies are provided in the Examples section below.

The following examples provide illustrative embodiments of the invention. One of ordinary skill in the art will recognize the numerous modifications and variations that may be performed without altering the spirit or scope of the present invention. Such modifications and variations are encompassed within the scope of the invention. The Examples do not in any way limit the invention.

### EXAMPLES

### Example 1

### MicroRNA Mimics

The microRNA mimic used in the following examples, and referenced above, is a synthetic mimic of miR-34a called MRX34. MRX34 comprises two complementary RNA molecules in a duplex structure. One RNA strand is an unmodified 23 mer with a sequence that is identical to miR-34a. The second RNA strand is a perfect complement to the first and is also unmodified except for a C₆-amine cap at the 5′ end of the RNA molecule. The 5′ cap on the complementary RNA prevents the molecule from functioning as a guide sequence for RNA-induced silencing complex (RISC) and thus ensures that MRX34 has the same functional activity as the endogenous miR-34a. miR-34 can be formulated in a liposomal injectable suspension, for example using Marina Biotech's SMARTICLES®.

### Example 2

### Summary of Non-Clinical Pharmacokinetics

Profiling of MRX34 included the characterization of the pharmacokinetic parameters and biodistribution following IV administration to the mouse, rat and non-human primate. Quantitative reverse-transcriptase real-time polymerase chain reaction (qRT-PCR) was used to measure the MRX34 oligonucleotide concentrations in total RNA isolated from whole blood and selected tissues.

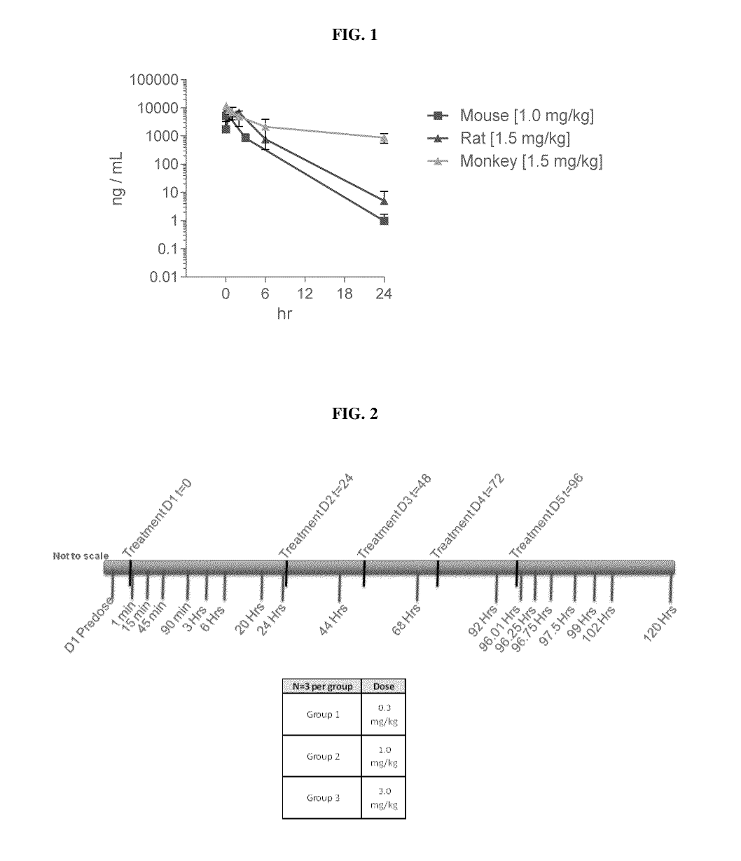

In pharmacokinetic studies, Balb/c mice, Sprague Dawley rats, and Cynomolgous monkeys received a single IV dose of MRX34. The dose levels used were 1 mg/kg (mouse) and 1.5 mg/kg (rat & non-human primate). At intervals after dosing, and at termination, blood samples were taken for the analysis of concentrations of MRX34. The blood concentrations of MRX34 (presented as copy number/ng of recovered RNA) are presented graphically in FIG. 1. Liposome-encapsulated MRX34 (MRX34) showed a long residence time in the blood with concentrations of miR-34a remaining above baseline at the last sampling points at 24 hours. Both rodent species show a comparable clearance rate of MRX34 in blood. In contrast, MRX34 shows a longer blood residence time in non-human primate. The estimated half-lives for formulated MRX34 in mouse, rat and monkey were 2.0, 2.2 and 7.9 hours, respectively. The estimated area-under-the-curve (AUC) values were 10,847, 35,869, and 65,126 ng*hr/mL, respectively. FIG. 1 illustrates blood levels of MRX34 (ng/mL of blood) as a function of time from administration.

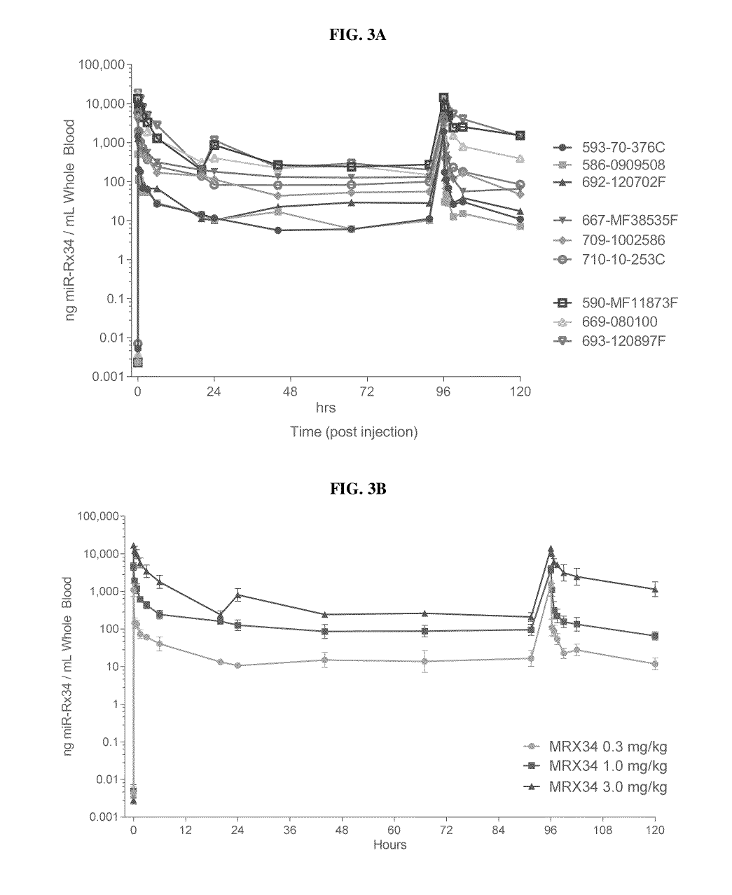

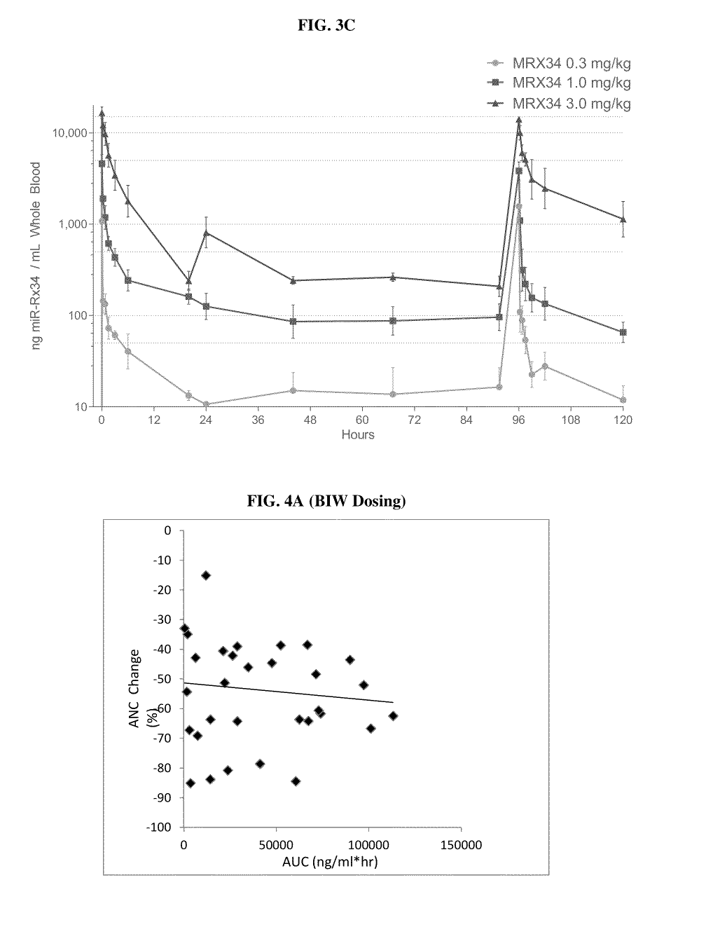

In another pharmacokinetic study monkeys received five consecutive daily IV doses of MRX34 in accordance with the invention. The monkeys were divided into three groups of three, with each group receiving a different daily dose. The timing of the dosing and composition of the groups is show in FIG. 2. FIGS. 3A-3C show the blood concentrations by animal (FIG. 3A) and by cohort (FIGS. 3B and 3C).

In a biodistribution study, Balb/c mice received a single IV dose of MRX34 at a dose level of 1 mg/kg. At intervals after dosing, and at termination, selected tissues were taken for the quantification of concentrations of MRX34. Among the tissues tested, liver and spleen displayed the longest residence time of MRX34. The level of MRX34 in the liver remained relatively constant throughout the evaluation period. There was more rapid clearance of MRX34 noted in the spleen and other tissues. At day 5 post administration, liver, spleen, adipose tissue and lung show MRX34 levels that are significantly elevated compared to endogenous miR-34a in these tissues. Cmax for most tissues is as early as 3 min, except liver (30 min) and spleen (180 min).

The SMARTICLES®-formulated MRX34 was rapidly cleared from the blood and showed accumulation in the liver and spleen. The blood concentration vs. time profile showed an unexpected decrease in concentration at the 15 minute point. The shape of the curve precluded any relevant curve fitting or estimation of pharmacokinetic parameters. The level of miR-34a in the liver remained relatively constant throughout the evaluation period. There was more rapid clearance of MRX34 noted in the spleen and other tissues.

### Example 3

### Summary of Non-Clinical Toxicology

MRX34 was evaluated in the mouse, rat, and non-human primate to identify potential drug related toxicities. In the non-GLP studies, rats and monkeys received MRX34 at dose levels of 1.5, 5, or 15 mg/kg/day administered every-other-day for fourteen days (7 doses). Control groups in these studies included dilution buffer and unloaded NOV340 SMARTICLES®. The GLP studies were conducted in the rat and non-human primate at dose levels of 3, 10, or 30 mg/kg/kg administered three times per week for four weeks (12 total doses).

There was no mortality or morbidity noted in any animals in the non-GLP studies. In the GLP studies, morbidity was seen in animals in the high dose group after 3-6 doses. These animals were euthanized and dosing was discontinued in this group. In the rat study, 2 animals were found dead and 2 animals were moribund after a single high dose. Dosing was discontinued in the high dose group.

The most consistent finding in all studies was a dose related decrease in platelets and an increase in spleen and liver weights. There were variable decreases in red blood cell count, haemoglobin, and hematocrit and increases in serum cholesterol. There was no evidence of cytokine stimulation or infusion reactions at any dose level tested. In the non-human primates, complement was depleted in the high dose animals following a single dose. Complement levels returned to baseline during dosing intervals and in the recovery animals. Histological observations included dose related increases in Kupffer cells in the liver and histiocytes in the spleen. The findings in the dose groups were similar to the findings in the unloaded NOV340 SMARTICLES® group and suggest that the toxicities are related to the clearance of the liposomes by the mononuclear phagocyte system. This clearance mechanism results in increased mononuclear cells in the liver and spleen and the subsequent sequestration of platelets in these organs. There was no evidence of bone marrow toxicity or any impact on platelet production or maturation.

Recovery of these toxicities was noted in the recovery animals in the GLP studies. The morbidity and deaths noted in the high dose groups in the non-human primate GLP study were considered to be related to a significant decrease in platelets that was associated with hemorrhaging. There was no conclusion on the cause of death in the rat study.

The main dose related toxicity noted in the animal studies, decreased platelets, is an easily monitored toxicity endpoint and the recovery data from the GLP studies show that platelet levels will return toward baseline with discontinuation of dosing.

### Example 4

### Side Effects, Dosing, and Premedication

Preliminary results from intravenous MRX34 treatment of eighteen patients with various solid tumors indicate that twelve patients experienced Grade 2 or less infusion-related reactions characterized mainly by fevers and chills, including shaking chills or rigors. These reactions began at various timepoints during and after the infusions and were controlled by interrupting or slowing the infusions and treating with acetaminophen/NSAID, with or without glucocorticoids. These patients were treated under a protocol prohibiting premedication.

Another protocol requires premedication with dexamethasone 10 mg IV prior to each treatment on Cycle 1 and Cycle 2, with the option of adding other premedications, such as acetaminophen, NSAID or a COX-2 inhibitor or an H1 or H2 blocker. Premedication on subsequent cycles can be at the discretion of the healthcare provider. In patients with cirrhosis, the risk-benefit of acetaminophen must be considered carefully. Similarly, in patients at risk of bleeding or renal compromise, the NSAID/COX-2 inhibitors must be used with care. With premedication, six patients on the 33 mg/m² and 50 mg/m² did not experience the same intensity of fever but had back pain thought secondary to the liposomal delivery system. Further dilution of the drug reduced the infusion related back pain.

### Example 5

### Effects on Myeloid Precursors

Some patients experienced brief Grade 3 or 4 neutropenia and/or thrombocytopenia after receiving MRX34. One patient experienced a recurrence of atrial flutter/fibrillation after having a febrile reaction to MRX34. One patient had G3 acute kidney injury which required hospitalization. There have been no episodes of hypotension in patients during or following IV infusion of MRX34. (Data not shown.)

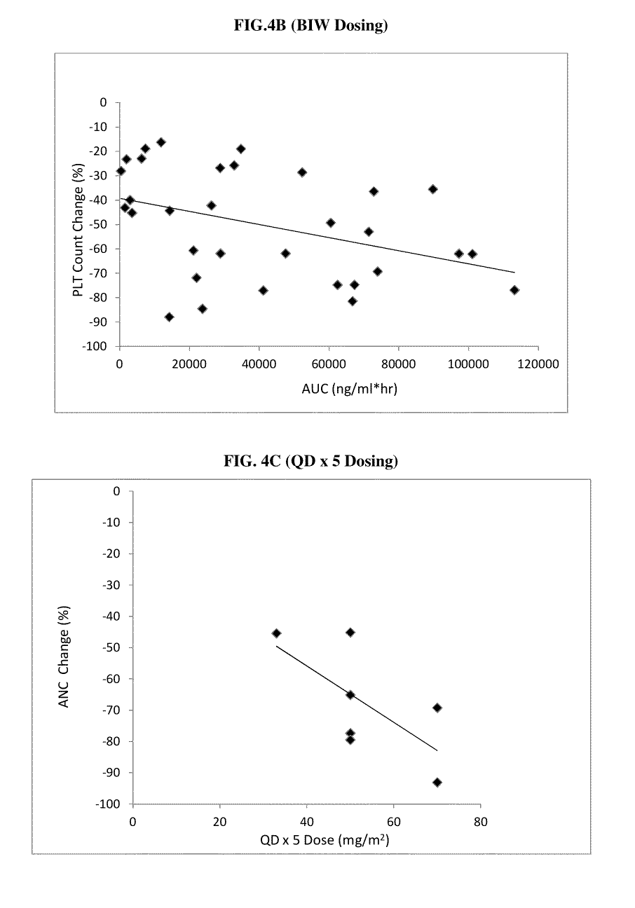

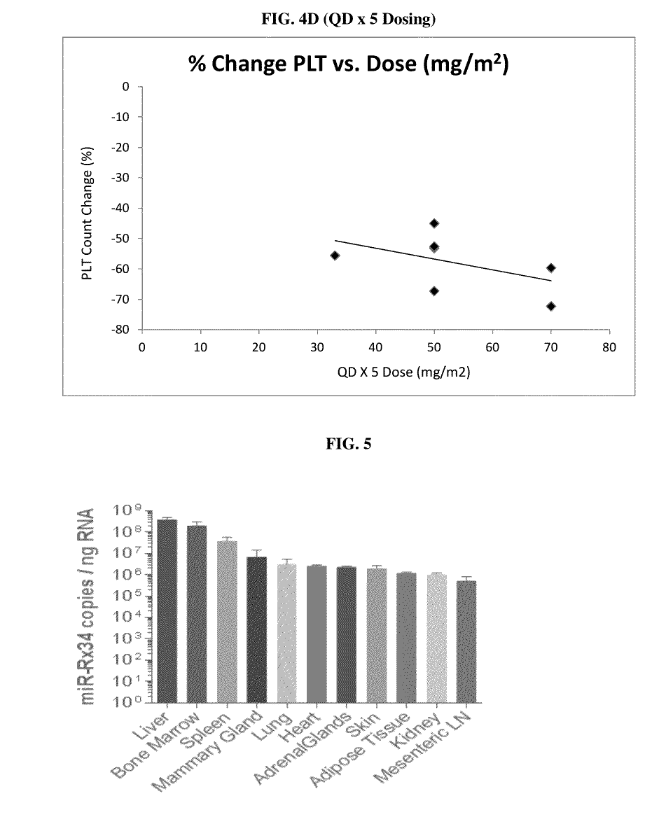

Preliminary analysis showed a correlation between nadir absolute neutrophil/platelet counts and MRX34 exposure as shown in FIGS. 4A and 4B, indicating a likely effect of MRX34 on human myeloid precursors. In addition, biodistribution studies in animals showed a high accumulation of MRX34 in bone marrow (FIG. 5).

### Example 6

### Treatment of a Subject Having a Hematologic Malignancy

First, a subject having a hematologic malignancy is selected for treatment. The hematologic malignancy can be, for example, non-Hodgkin's lymphoma (NHL), Hodgkin's lymphoma (HL), acute myeloid leukemia (AML), acute lymphocytic leukemia (ALL), lymphoma, chronic lymphocytic leukemia (CLL), multiple myeloma (MM), myelodysplastic syndrome (MDS) and chronic myeloid leukemia (CML) in accelerated or blast phase.

Approximately 12 hours prior to the first dose of MRX34 dexamethasone 10 mg PO will be started as premedication. On Days 1-5 of each cycle, MRX34 infusion over 2 hours will be administered daily. Dexamethasone 10 mg PO bid (every 12 hours) will be administered on Days 1, 2, 3, 4, 5, 6 and 7 of every cycle to minimize infusion-related reactions. See Table 4A. Additional doses of dexamethasone and/or other premedications (such as H1 or H2 blockers) can be administered per treating physician's clinical judgment, to minimize infusion-related reactions. In alternative embodiments, dexamethasone and/or other premedication can be omitted.

One cycle will be defined as 21 days. The MRX34 dose can be 20, 33, 50, 70, or 93 mg/m² daily for 5 days. However, under no circumstance will any single dose exceed 10 mg/kg (˜370 mg/m², based upon monkey and rat toxicology). For hematologic malignancy patients, the starting dose will be 33 mg/m² daily×5 (a total of 100 mg/m² over 5 days) in 21-day cycles. See Table 4A. Adjustment to the starting dose can be made within this range.

MRX34 is provided as a 20 mL vial with a 15 mL fill of 3 mg/mL for a total of 45 mg per vial. The product must be kept frozen in a −20° C. freezer and will be shipped on dry ice with a temperature monitoring system. The study drug will be thawed at room temperature on the day of preparation or in the refrigerator overnight. The infusion should be prepared within 90 minutes of removal from freezer or refrigerator. The study drug will be withdrawn from the vial and mixed in 100 to 250 mL of Normal Saline. The preferred solution for infusion is Normal Saline; however should the patient have a medical condition that precludes the use of Normal Saline then D5W may be used. The product should be completely administered to the patient within 4 hours after infusion preparation. The product should be refrigerated if not administered within 1 hour of the infusion preparation. The drug should be infused without filtration through a controlled infusion pump over approximately 2 hours. This infusion schedule may be revised based on patient responses and practical considerations of infusing various volumes of reconstituted MRX34. microRNA preparation can vary, for example depending upon formulation and route of administration.

Subjects who successfully complete treatment Cycle 1 (21 days) without evidence of significant treatment-related toxicities or clinical evidence of progressive disease can continue receiving treatment. Treatment cycles can be repeated every 3 weeks (21 days) for hematologic malignancy patients based on toxicity and response. The schedule can continue as long as there is perceived benefit or until clinically significant disease progression. The efficacy of treatment can be assessed by the clinically accepted Response Criteria for the particular indication. Such Response Criteria are well known in the art, and can be applied in the various embodiments of the invention.

TABLE 4A
MRX34 Dosing Schedule 5 × Daily Every 21 days

| | Dosing Period | | | | | | | | | | |
|---|---|---|---|---|---|---|---|---|---|---|---|
| | | | | | | | | | Cycle 2 and | | |
| | Cycle 1 | | | | | | | | subsequent cycles | | |
| | Dosing Day | | | | | | | | | | |
| | 12 | | | | | | | | | | |
| | hours | | | | | | | | Days | | |
| | prior | | | | | | | | 1, 2, | | |
| | to | | | | | | Days | Days | 3, | Days | Days |
| | Day 1 | Day 1 | Day 2 | Day 3 | Day 4 | Day 5 | 6, 7 | 8-21 | 4, 5 | 6, 7 | 8-21 |
| Dexamethasone | x | | | | | | | | | | |
| 10 mg PO | | | | | | | | | | | |
| Dexamethasone | | x | x | x | x | x | x | | x | x | |
| 10 mg PO bid | | | | | | | | | | | |
| MRX34 | | x | x | x | x | x | | | x | | |
| infusion | | | | | | | | | | | |

MRX34 infusion will be given once per day for 5 days followed by 2 weeks rest (total of 5 doses per 21 day cycle every cycle). 12 hours prior to Cycle 1 Day 1 premedicate with dexamethasone 10 mg PO. Dexamethasone 10 mg PO will be given Days 1 through 7 of every cycle.

### Example 7

### Treatment of a Subject Having a Solid Tumor

First, a subject having a solid tumor is selected for treatment. The solid tumor can be, for example, HCC, small-cell lung cancer, non-small cell lung cancer, neuroendocrine tumor, colon cancer, breast cancer, melanoma, or renal cell carcinoma.

Approximately 12 hours prior to the first dose of MRX34 dexamethasone 10 mg PO will be started as premedication. On Days 1-5 of each cycle, MRX34 infusion over 2 hours will be administered daily. Dexamethasone 10 mg PO bid (every 12 hours) will be administered on Days 1, 2, 3, 4, 5, 6 and 7 of every cycle to minimize infusion-related reactions. See Table 4A. Additional doses of dexamethasone and/or other premedications (such as H1 or H2 blockers) can be administered per treating physician's clinical judgment, to minimize infusion-related reactions. In alternative embodiments, dexamethasone and/or other premedication can be omitted.

One cycle will be defined as 21 days. The MRX34 dose can be 20 mg/m² daily for 5 days. However, under no circumstance will any single dose exceed 10 mg/kg (˜370 mg/m²). For hematologic malignancy patients, the starting dose will be 20 mg/m² daily×5 (a total of 100 mg/m² over 5 days) in 21-day cycles. See Table 4A. Adjustment to the starting dose can be made within this range. MRX34 can be prepared as described in Example 6 above.

Subjects who successfully complete treatment Cycle 1 (21 days) without evidence of significant treatment-related toxicities or clinical evidence of progressive disease can continue receiving treatment. Treatment cycles can be repeated every 3 weeks (21 days) for solid tumor (e.g., HCC) patients based on toxicity and response. The schedule can continue as long as there is perceived benefit or until clinically significant disease progression. The efficacy of treatment can be assessed by the clinically accepted Response Criteria for the particular indication. Such Response Criteria are well known in the art, and can be applied in the various embodiments of the invention.

### Example 8

### Comparison of Adverse Events in Twice Weekly and Daily×5 Administration of MRX34

For patients receiving twice weekly (BIW) dosing of MRX34, patients are usually treated on Mondays and Thursdays for 3 weeks and then given a rest from treatment for 1 week. Each cycle is defined as 4 weeks. Table 4B shows the BIW dosing schedule schematically:

TABLE 4B
BIW dosing schedule

| | Dosing Period | | | | | |
|---|---|---|---|---|---|---|
| | | | | Cycle 2 and | | |
| | Cycle 1 | | | subsequent cycles | | |
| | Dosing Day | | | | | |
| | Day | Day | Days | Days | Days | Days |
| | 1, 4 | 8, 11 | 15, 18 | 1, 4 | 6, 7 | 8-21 |
| Dexamethasone | x | x | x | x | x | x |
| 10 mg IV just | | | | | | |
| before MRX34 | | | | | | |
| MRX34 infusion | x | x | x | x | x | x |

Dexamethasone appears to be helpful in managing the infusion reactions. However, it was impractical to give dexamethasone continuously for 3 weeks to cover the entire dosing period of each cycle since such a prolonged dexamethasone would cause undue toxicities including adrenal gland suppression, osteoporosis, and increased risks of infections. Therefore, dexamethasone was given as a single dose just before each infusion for twice weekly MRX34 dosing regimen. In contrast, with the daily×5 dosing (see Table 4A above), it would be feasible to give dexamethasone continuously to cover the entire week of dosing, which reduces the infusion reactions associated with MRX34 without causing undue toxicities from chronic continuous steroid administration.

Table 4C presents patient characteristics of the 71 patients enrolled on this study which includes 47 patients treated BIW and 24 treated QD×5. The majority of patients had an ECOG performance of 1 on enrollment; 60% were white; the median number of prior therapies for patients enrolled in the biweekly arm was 4; the most frequent enrolled tumor types are Hepatocellular cancer, pancreatic cancer and cholangiocarcinoma.

TABLE 4C
Patient Characteristics (N = 71)

| | BIW | QD × 5 |
|---|---|---|
| | (n = 47) | (n = 24) |
| Median age (range); | 60 (29-86); | 60 (33-78); |
| Males (%) | 26 (57) | 17 (71) |
| ECOG Performance Score 0/1/2 (%) | 28/65/7 | 0/91/9 |
| Race: White/Hisp/Blk/Asian/other (%); | 59/15/11/11/4 | 67/0/4/29/0 |
| Prior Therapy: Median number | 4 | 4 |
| Cancer Type | | |
| Hepatocellular carcinoma | 14 | 8 |
| Pancreatic ca. | 5 | — |
| Cholangiocarcinoma | 4 | — |
| Neuroendocrine tumor | 3 | 2 |
| Colorectal, breast, cervical ca. | 3, 3, 3 | — |
| Leiomyosarcoma, | 2, 2, 2, 0, 0, 0 | 0, 0, 1, 3, 4, 1 |
| bladder, esophageal, | | |
| Hodgkin's, AML, MDS | | |
| Other (1 each) | 6* | 5** |

*Adenocarcinoma of unknown primary, appendiceal adenocarcinoma, ovarian, GIST, NSCL, and pheochromocytoma
**Small cell lung cancer, diffuse large B-cell lymphoma, NK/T-cell lymphoma, prostate cancer, and apocrine adenocarcinoma

Table 5 below presents data showing that a Daily×5 regimen (also referred to a D×5 or QD×5, see Table 4A and Examples 6 and 7 above) has fewer adverse events than a Twice Weekly regimen (also referred to a BIW, see Table 4B).

Results from twice weekly intravenous MRX34 treatment of 47 patients with various solid tumors indicate that the most frequent adverse events have been the infusion reactions such as fever, chill, back pain, nausea, vomiting and diarrhea (Table 5). These reactions began at various timepoints during and after the infusions and were managed by interrupting or slowing the infusions and treating with acetaminophen/NSAID, with or without glucocorticoids. A majority of patients also experienced back pain, which is thought to be secondary to the liposomal delivery system. Further dilution of the study drug in a larger infusion volume appeared to reduce the infusion-related back pain. Other common adverse events include fatigue, dehydration, dysgeusia, and headache. Two patients experienced atrial flutter/fibrillation after having a febrile reaction to MRX34. Both patients experienced rapid resolution of the atrial fibrillation.

In contrast, results from QD×5 intravenous MRX34 treatment of 24 patients indicate a reduced incidence of infusion-related reactions at 33, 50 or 70 mg/m² daily×5 when administered with dexamethasone 10 mg BID×7 days, starting 12 hours before the first dose. See Table 5. Fewer patients had fever, chills, back pain, nausea, vomiting and diarrhea of all grades, as well as fewer grade 3 (G3) fatigue, back pain, diarrhea and abdominal pain. Two patients with refractory AML with pre-existing severe, prolonged neutropenia on multiple antibiotics for prior sepsis developed sepsis again on study, which was not unexpected.

TABLE 5
Most Common Adverse Events, Twice Weekly Regimen &
Daily × 5 Regimen* (N = 71)

| | BIW (n = 47) | | | QD × 5 (n = 24) | | |
|---|---|---|---|---|---|---|
| | All | | | All | | |
| Adverse | Grades | G3 | G4 | Grades | G3 | G4 |
| Event** | n (%) | n (%) | n (%) | n (%) | n (%) | n (%) |
| Fever | 34 (72) | 1 (2) | — | 9 (38) | — | — |
| Fatigue | 27 (57) | 5 (11) | — | 9 (38) | — | — |
| Chills | 26 (55) | — | — | 8 (33) | — | — |
| Back Pain | 23 (49) | 5 (11) | — | 5 (21) | — | — |
| Nausea | 23 (49) | 1 (2) | — | 5 (21) | — | — |
| Diarrhea | 20 (43) | 5 (11) | — | — | — | — |
| Vomiting | 14 (30) | 2 (4) | — | 1 (4) | — | — |
| Headache | 14 (30) | — | — | 6 (25) | — | — |
| Anorexia | 12 (26) | — | — | 2 (8) | — | — |
| Dehydration | 11 (23) | 3 (6) | — | — | — | — |
| Abdominal | 10 (21) | 4 (9) | — | 3 (13) | 1 (4) | — |
| pain | | | | | | |
| Insomnia | 10 (21) | — | — | 3 (13) | — | — |
| Dyspnea | 10 (21) | — | — | 1 (4) | — | — |
| Dysgeusia | 8 (17) | — | — | 2 (8) | — | — |
| Constipation | 8 (17) | — | — | 2 (8) | — | — |

*Administered with dexamethasone premedication, 10 mg PO or IV, BID × 7 days, starting 12 hours before the first MRX34 dose;
**Adverse events >15%.

### Example 9

### Comparison of Laboratory Abnormalities in Twice Weekly and Daily×5 Administration of MRX34

Tables 6 and 8 present data showing treatment-emergent≧Grade 2 chemistry laboratory abnormalities. Most of the patients with elevated alanine transaminase (ALT), aspartate transaminase (AST), or bilirubin had concurrently elevated alkaline phosphatase, consistent with progressive liver metastasis or primary liver cancer. Two patients with HCC and large liver lesions developed Grade 4 elevations in AST and/or ALT within 4 days of receiving the first dose of MRX34 at 50 mg/m² BIW dose level. The elevations in AST/ALT resolved over the next 2 weeks. Neither of the two patients had concurrently elevated bilirubin and both patients subsequently received additional MRX34 doses without recurrence of Grade 4 AST/ALT elevations. It was thought that these patients with HCC may have had transaminase releases from malignant or transformed hepatocytes rather than from normal hepatocytes. One of the patients had an increased uric acid along with the elevation in AST/ALT and the other patient received prophylactic allopurinol before the first dose. To prevent complications of potential tumor lysis syndrome, as tolerated by the patient, aggressive hydration should be maintained after each dose, especially if the patient is experiencing decreased oral fluid intake, nausea, vomiting or diarrhea.

One patient developed Grade 3 acute kidney injury with elevated creatinine (G3) following the first dose of MRX34 at 20 mg/m² twice weekly. This patient had NSCLC, previously treated with carboplatin-containing regimen and then cisplatin-containing regimen with a history of creatinine elevation. After the first dose of study drug, the patient developed nausea, vomiting, diarrhea, light-headedness and then experienced the acute kidney injury, which resolved after 2 weeks. This event was deemed to be a DLT.

Most of the patient receiving QD×5 dosing regimen developed hyperglycemia while receiving dexamethasone as premedication. Blood glucose was monitored frequently and some patients received insulin or oral hypoglycemic agents to manage hyperglycemia.

TABLE 6
Chemistry Laboratory Abnormalities, BIW vs. QDX5 Regimens (N = 71)

| | BIW Regimen (n = 47) | | | QD × 5 Regimen (n = 24) | | |
|---|---|---|---|---|---|---|
| Lab | G2 | G3 | G4 | G2 | G3 | G4 |
| Abnormality | n (%) | n (%) | n (%) | n (%) | n (%) | n (%) |
| Albumin↓ | 20 (43) | 2 (4) | — | 6 (25) | — | — |
| Alk Phos | 9 (19) | 5 (11) | — | 4 (17) | — | — |
| ALT↑ | 3 (6) | 3 (6) | 1 (2) | 1 (4) | 5 (21) | |
| AST↑ | 6 (13) | 9 (19) | 2 (4) | 4 (17) | 3 (13) | — |
| Bilirubin↑ | 5 (11) | 3 (6) | — | 2 (8) | — | — |
| Creatinine↑ | 1 (2) | 1 (2)* | — | — | — | — |
| Glucose↑ | 4 (9) | 7 (15) | 1 (2) | 10 (42) | 4 (17) | 1 (4) |
| Lipase↑ | 3 (6) | — | 1 (2) | — | 2 (8) | — |
| Sodium↓ | — | 11 (23) | 2 (4) | — | 2 (8) | — |

*DLT

TABLE 7
Hematology Laboratory Abnormalities (N = 59)

| | BIW Regimen (n = 47) | | | QD × 5 Regimen (n = 12) | | |
|---|---|---|---|---|---|---|
| Lab | G2 | G3 | G4 | G2 | G3 | G4 |
| Abnormality | n (%) | n (%) | n (%) | n (%) | n (%) | n (%) |
| Anemia | 16 (34) | 5 (11) | — | 7 (29) | — | — |
| Leukopenia | 16 (34) | 9 (19) | 1 (2) | 7 (29) | 3 (13) | — |
| Lympho- | 12 (26) | 11 (23) | 6 (13) | 1 (4) | 7 (29) | 5 (21) |
| penia | | | | | | |
| Platelet↓ | 10 (21) | 8 (17) | — | 6 (25) | 4 (17) | 4 (17) |
| Neutropenia | 8 (17) | 7 (15) | 4 (9) | 2 (8) | 2 (8) | 3 (13) |

### Example 10

### Comparison of Pharmacokinetics in Twice Weekly and Daily×5 Administration of MRX34

FIGS. 6A-B present data showing the whole-blood pharmacokinetic profile of twice weekly MRX34 at various dose levels and Table 8 shows the PK parameters at various doses of MRX34 administered twice weekly. As doses are increased, the C. and AUC increased non-linearly. The terminal half was >1 day in general.

TABLE 8
Pharmacokinetic Parameters, Twice Weekly Dosing

| Dose | | Cₘₐₓ (ng/ml) | | AUCₗₐₛₜ (hr * ng/ml) | | T_{1/2} (hr) | |
|---|---|---|---|---|---|---|---|
| (mg/m²⁾ | N | Day 1 | Day 18 | Day 1 | Day 18 | Day 1 | Day 18 |
| 10 | 3 | 222 ± 64 | 224 ± 84 | 3210 ± 2650 | 993 ± 1080 | 31 ± 7 | 64 ± 24 |
| 20 | 6 | 712 ± 564 | 1060 ± 592 | 7310 ± 6630 | 8860 ± 5320 | 35 ± 11 | 67 ± 54 |
| 33 | 3 | 1570 ± 276 | 1210 ± 154 | 20400 ± 8610 | 8870 ± 4890 | 32 ± 1 | 33 ± 20 |
| 50 | 6 | 4050 ± 871 | 3160 ± 1690 | 54700 ± 22500 | 35300 ± 16200 | 22 ± 6 | 31 ± 12 |
| 70 | 9 | 5940 ± 1560 | 1950 | 51520 ± 24450 | 17180 | 28 ± 12 | 26 |
| 93 | 4 | 7120 ± 4200 | 3420 | 72100 ± 34350 | 54080 | 29 ± 8 | 41 |
| 110 | 3 | 4580 ± 3760 | 3870 | 48500 ± 37600 | 16542 | 25 | 8 |
| 124 | 2 | 20560 | — | 92500 | — | — | — |

FIG. 7 shows the whole-blood pharmacokinetic profile of daily×5 MRX34 and Table 9 shows the PK parameters at various doses of MRX34 administered daily×5. With the daily dosing regimen, there was accumulation over the 5 days with the AUC on Day 5 being much greater than that on Day 1, resulting in higher drug exposure compared to twice weekly dosing. Terminal half-life was longer as well compared to twice weekly dosing regimen.

TABLE 9
Pharmacokinetic Parameters, Daily × 5 Dosing

| Dose | | Cₘₐₓ (ng/ml) | | AUCₗₐₛₜ (hr * ng/ml) | | T_{1/2} (hr) | |
|---|---|---|---|---|---|---|---|
| (mg/m²⁾ | N | Day 1 | Day 5 | Day 1 | Day 5 | Day 1 | Day 5 |
| 33 | 1 | 662 | 2290 | 11700 | 179000 | NA* | 57 |
| 50 | 4 | 2800 ± 1010 | 5800 ± 1500 | 23600 ± 10300 | 330000 ± 156000 | NA | 53 ± 17 |
| 70 | 4 | 4800 ± 1800 | 12400 ± 2600 | 31700 ± 8500 | 427000 ± 218000 | NA | 38 ± 12 |

*NA, not available

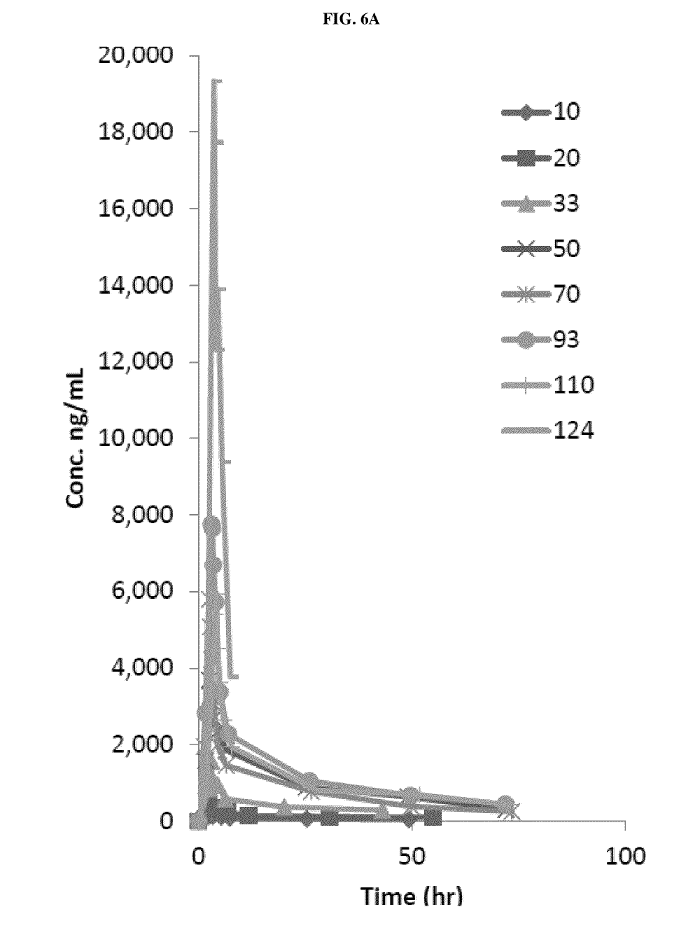

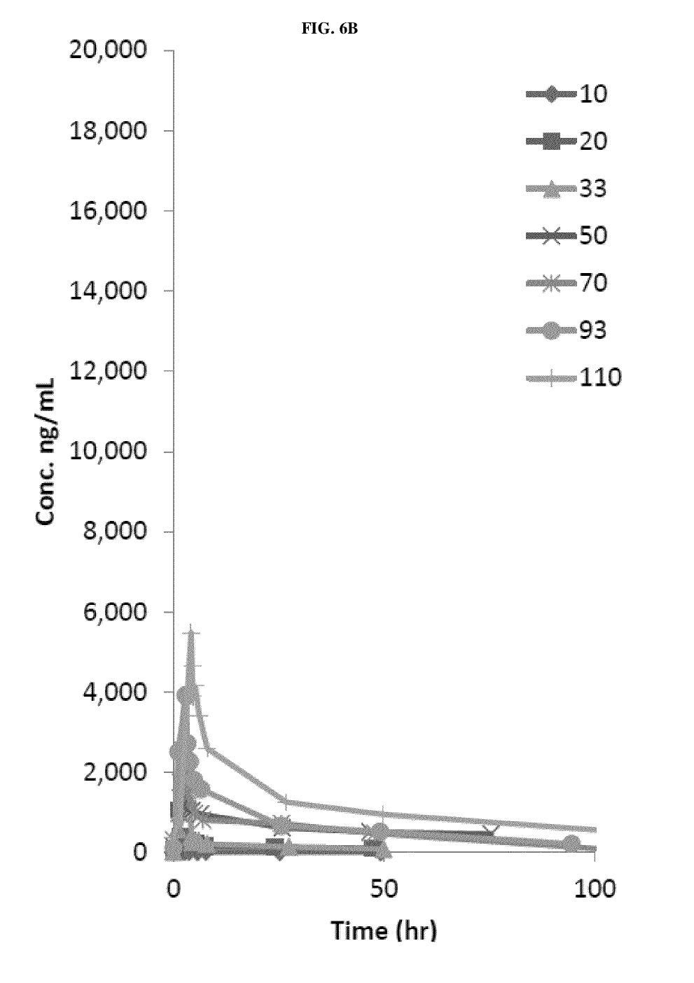

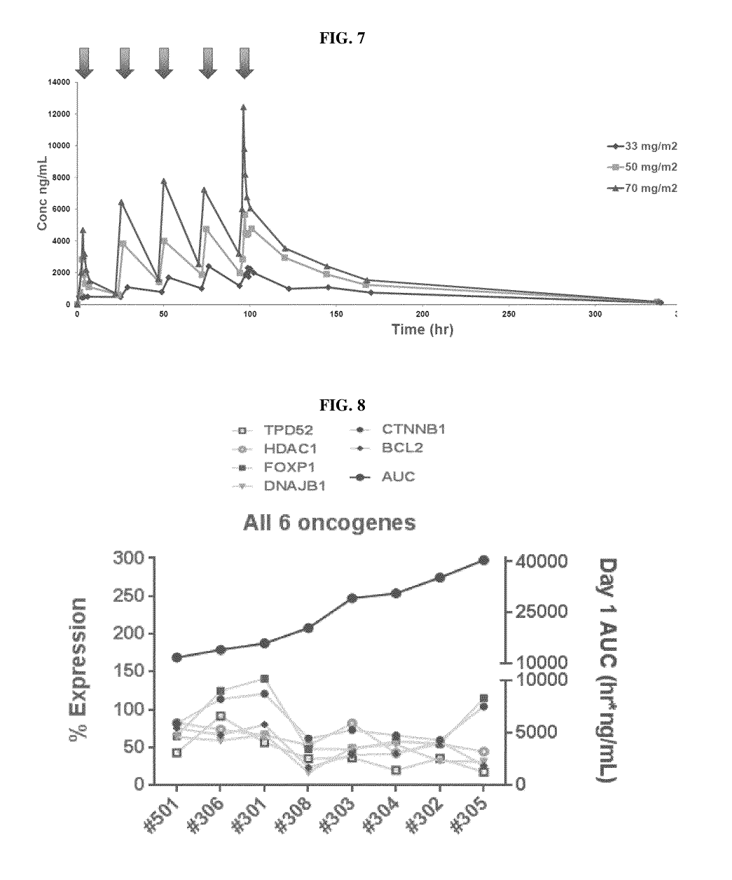

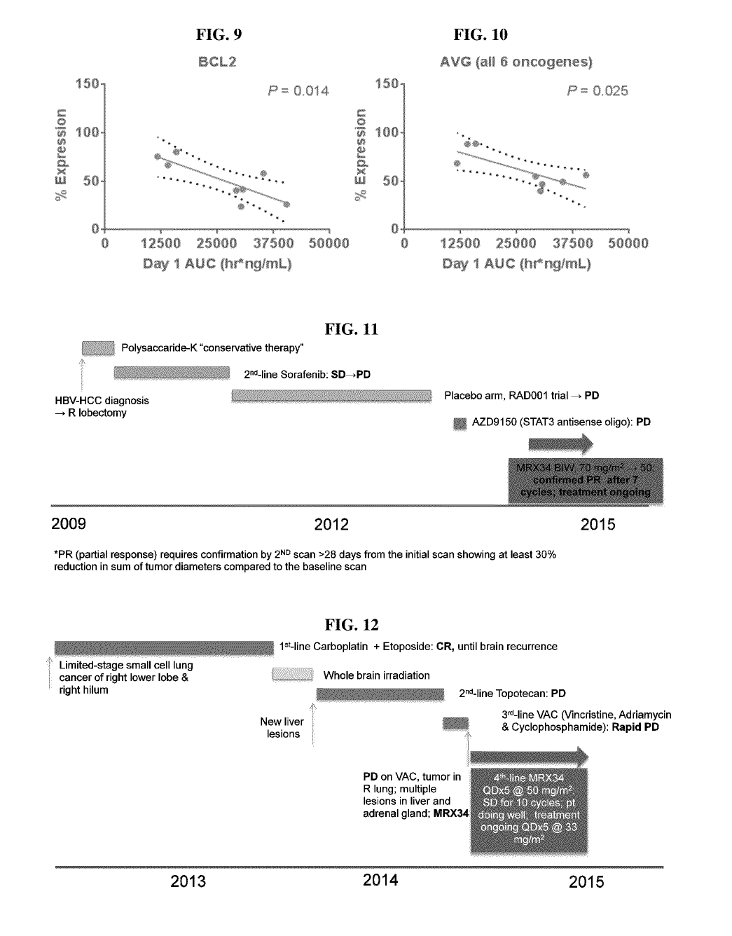

FIGS. 8-10 present white blood cell gene expression for selected subjects 24 hours after first infusion with MRX34. FIG. 10 presents the % expression and area under curve (AUC) for six oncogenes in 8 different subjects (#501, 306, 301, 308, 303, 304, 302, and 305). FIG. 11 presents the % expression vs. AUC in these subjects for the BCL2 oncogene. FIG. 12 presents the % expression vs. AUC averaged (AVG) over all six oncogenes.

FIG. 11 presents the results for a subject showing confirmed partial response (confirmation by 2ND scan>28 days from the initial scan showing at least 30% reduction in sum of tumor diameters compared to the baseline scan) for HBV (hepatitis B virus)-HCC. The subject had prolonged stable disease for 16 months on sorafenib; rapid progressive disease after two months on AZD9150 (STAT3 antisense oligonucleotide); initial stable disease for several cycles of BIW MRX34 and 30% tumor size reduction after cycle 6.

FIG. 12 presents the results for a subject showing prolonged stable disease in heavily pretreated SCLC. The subject had progressive disease after three courses of chemotherapy, started QD×5 MRX34 as a fourth line therapy, and has had stable disease for 10 cycles (treatment ongoing).

Other patients have also experienced positive results. For example, patient #501 had DLBCL, was placed on 50 mg/m2 QD×5, and achieved complete resolution of lymphoma after 2 cycles. Table 15 below presents the clinical history of patient #501.

TABLE 15
Clinical history of patient #501.

| | |
|---|---|
| 2011 | 78-year-old male with high grade diffuse large B-cell lymphoma KI-67 |
| | 80%, CD19 and 20 positive, CD5 negative. Occipital lytic lesion, several |
| | infratemporal fossa and rt. posterior hypopharynx wall and piriformis sinus. |
| | Significant weight bearing osseous disease in femurs |
| 11 Sep 2011 | R2-CHOP, relapsed with skin lesions - radiation June 2012. |
| March 2013 | ISIS antisense STATS trial March 2013 |
| 27 May 2014 | Baseline PET - medial left lower leg lesions and lateral distal lower leg |
| | deoxyglucose uptake, right inguinal area lesion - negative biopsy. CT scan- |
| | multiple skin lesions left lower leg 1.5 to 3 cm in size |
| 02 Jun 2014 | Patient enrolled into MRX34 study (50 mg/m² dose QD × 5) |
| 14 Jul 2014 | PET - complete resolution of PET uptake of lower leg that showed |
| | lymphoma on previous biopsy. Continued to show uptake in right inguinal |
| | area previously negative biopsy. Per Cheson criteria, potentially complete |
| | response taking into account the R inguinal node with PET uptake showed |
| | no lymphoma on previous biopsy. Clinic note also indicates that the patient |
| | and wife were told that the patient has achieved complete response. |

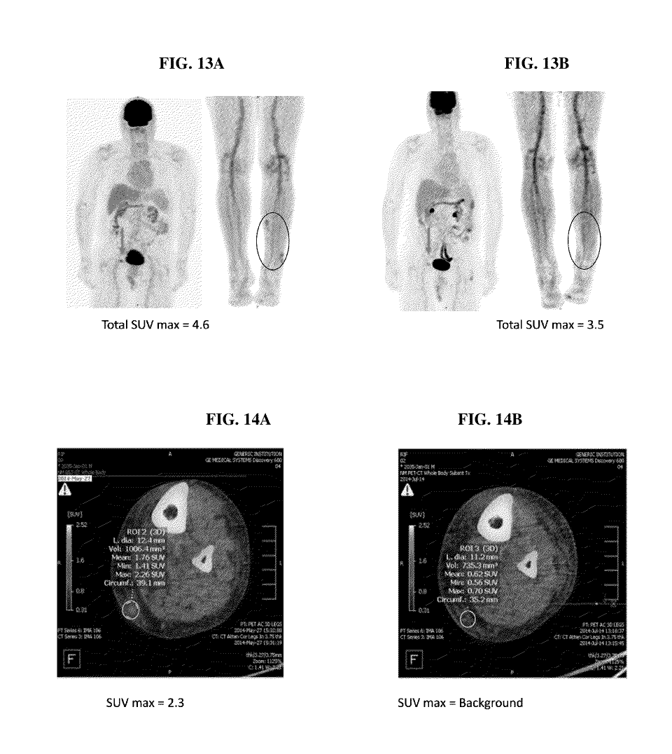

FIG. 13A (baseline) and FIG. 13B (after cycle 2) present PET/CT scans after 2 cycles of, 33 mg/m² QD×5 MRX34. The arrows show PET positive activity in the right groin. The ovals show a previous biopsy of the right inguinal node, which was negative and thought to be non-lymphoma.

FIG. 14A (baseline) and FIG. 14B (after cycle 2) present PET/CT scans after 2 cycles of, 33 mg/m² QD×5 MRX34 at the patients lower left extremity (LLE)—the visible skin nodules in the LLE, previously biopsy positive for lymphoma, also resolved after 2 cycles.

PET activity in left lower extremity (LLE) that completely resolve after 2 cycles

In summary, it was found that BIW dosing has a MTD 110 mg/m², with a manageable safety profile with dexamethasone premedication and a non-linear PK, half-life>1 day. These data also show that the QD×5 dosing has a higher drug exposure on D5 vs. D1. MTD was not reached for QD×5. Furthermore, these data establish that MRX34 has shown target repression in human WBCs and that MRX34 has activity in HCC, SCLC, and heme malignancies.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. Those skilled in the art will recognize, or be able to ascertain using no more than routine experimentation, many equivalents to the specific embodiments of the invention described herein. Such equivalents are intended to be encompassed by the following claims.

### REFERENCES

1. Iorio M C, Croce C M. MicroRNA involvement in human cancer. Carcinogenesis 2012; 33:1126-1133.
2. Nikitina E G, Urazova L N, Stegny V N. MicroRNAs and human cancer. Exp Oncol 2012; 34:2-8.
3. Schoof C R G, da Silva Botelho E L, Izzotti A, dos Reis Vasques L. MicroRNAs in cancer treatment and prognosis. Am J Cancer Res 2012; 2:414-433.
4. Croce C. MicroRNAs in leukemia. Clin Adv Hematol Oncol 2006; 4:577-578.
5. Garzon R, Fabbri M, Cimmino A, Calin G A, Croce C M. MicroRNA expression and function in cancer. Trends Mol Med 2006; 12:580-587.
6. Hammond S M. RNAi, microRNAs, and human disease. Cancer Chemother Pharmacol 2006; 58 (Suppl 1):s63-68.
7. Hermeking H. The miR-34 family in cancer and apoptosis. Cell Death Differ 2010; 17:193-199.
8. Wong M Y W, Yu Y, Walsh W R, Yang J-L. microRNA-34 family and treatment of cancers with mutant or wild-type p53 (review). Int J Oncol 2011; 38:1189-1195.
9. Bader AG. miR-34—a microRNA replacement therapy is headed to the clinic. Front Genet 2012; 3:120.
10. Harb W, Lakhani N, Logsdon A, et al. BCL2 targeted deoxyribonucleic acid inhibitor (DNAi) PNT2258 is active in patients with relapsed or refractory non-Hodgkin's Lymphoma [abstract]. 55th ASH Annual Meeting and Exposition, 2013 Dec. 7-10; Abstract #88, New Orleans, La.
11. Ramanathan R, Hamburg S, Halfdanarson T, Borad M. A Phase I/II dose escalation study of TKM-080301, a RNAi therapeutic directed against PLK1, in patients with advanced solid tumors, with an expansion cohort of patients with NET or ACC. North American Neuroendocrine Tumor Society; Neuroendocrine Tumor Symposium, 2013 Oct. 4-5; Abstract #C31, Charleston, S.C.
12. Liu C, Kelnar K, Liu G, et al. The microRNA miR-34a inhibits prostate cancer stem cells and metastasis by directly repressing CD44. Nat Med. 2011; 17(2):211-215.

1. A method of administering a microRNA to an individual in need thereof, comprising: administering to the individual a therapeutically-effective amount of the microRNA for 3-7 consecutive days, followed by 7-21 consecutive days without administering the microRNA.

2. The method of claim 1, wherein the individual has a cancer.

3. The method of claim 2, wherein the cancer is a solid cancer.

4. The method of claim 2, wherein the cancer is a hematologic malignancy.

5. The method of claim 1, wherein the microRNA is formulated in a liposomal injectable suspension.

6. The method of claim 1, wherein the microRNA is selected from the group consisting of: a miR-34a mimic, miR-34b mimic, miR-34c mimic, miR-449a mimic, miR-449b mimic, and miR-449c mimic.

7. The method of claim 1, wherein the therapeutically-effective amount of the microRNA is administered to the individual for 5 consecutive days, followed by 16 consecutive days without administration of the microRNA.

8. (canceled)

9. The method of claim 1, further comprising administering a therapeutically effective amount of a glucocorticoid.

10. The method of claim 9, wherein the glucocorticoid is selected from the group consisting of: dexamethasone, hydrocortisone, cortisone, prednisone, prednisolone, methylprednisolone, betamethasone, triamcinolone, beclometasone, fludrocortisone acetate, deoxycorticosterone acetate, and aldosterone.

11. The method of claim 10, wherein the therapeutically effective amount of dexamethasone is 10 mg BID.

12. (canceled)

13. The method of claim 1, further comprising administering a therapeutically effective amount of a glucocorticoid on the 3-7 consecutive days that the microRNA is administered.

14. The method of claim 1, further comprising administering a therapeutically effective amount of a glucocorticoid for 1-5 days after administration of the microRNA.

15. The method of claim 1, further comprising administering a therapeutically effective amount of a glucocorticoid for 1-3 days before administration of the microRNA.

16. (canceled)

17. (canceled)

18. (canceled)

19. (canceled)

20. (canceled)

21. (canceled)

22. (canceled)

23. (canceled)

24. (canceled)

25. The method of claim 1, further comprising administering a therapeutically effective amount of an immunosuppressive agent.

26. The method of claim 1, wherein the microRNA is administered once per day.

27. The method of claim 1, wherein the therapeutically-effective amount of the microRNA is administered to the individual for 3 consecutive days, followed by 18 consecutive days without administration of the microRNA.

28. The method of claim 13, wherein the glucocorticoid is administered 12 hours prior to first administration of the microRNA.

28. The method of claim 3, wherein the solid cancer is selected from the group consisting of: a lung cancer, a liver cancer, a colon cancer, a pancreatic cancer, a breast cancer, a prostate cancer, a neuroendocrine tumor, a renal cell carcinoma, and a melanoma.

29. The method of claim 28, wherein the liver cancer is hepatocellular carcinoma.

30. the method of claim 28, wherein the lung cancer is non-small cell lung cancer (NSCLC).

31. The method of claim 4, wherein the hematological malignancy is selected from the group consisting of: a leukemia, a lymphoma, and a myeloma.

32. The method of claim 4, wherein the hematological malignancy is selected from the group consisting of: non-Hodgkin's lymphoma (NHL), Hodgkin's lymphoma (HL), acute myeloid leukemia (AML), acute lymphocytic leukemia (ALL), lymphoma, chronic lymphocytic leukemia (CLL), multiple myeloma (MM), myelodysplastic syndrome (MDS) and chronic myeloid leukemia (CML).


SEQUENCE LISTING (9 sequences)

SEQ ID NO: 1 - RNA, 23 nt, Homo sapiens
uggcagugucuuagcugguuguu

SEQ ID NO: 2 - RNA, 23 nt, Homo sapiens
uaggcagugucauuagcugauug

SEQ ID NO: 3 - RNA, 23 nt, Homo sapiens
aggcaguguaguuagcugauugc

SEQ ID NO: 4 - RNA, 22 nt, Homo sapiens
  misc_feature (1)..(1): n denotes a deletion or any nucleotide(s)
nggcagugunuuagcugnuugn

SEQ ID NO: 5 - RNA, 22 nt, Homo sapiens
uggcaguguauuguuagcuggu

SEQ ID NO: 6 - RNA, 22 nt, Homo sapiens
aggcaguguauuguuagcuggc

SEQ ID NO: 7 - RNA, 25 nt, Homo sapiens
uaggcaguguauugcuagcggcugu

SEQ ID NO: 8 - RNA, 22 nt, Homo sapiens
  misc_feature (14)..(14): n denotes a deletion or any nucleotide(s)
uggcaguguauugnuagcngng

SEQ ID NO: 9 - RNA, 7 nt, Homo sapiens
ggcagug